(19) United States
(12) Patent Application Publication
(10) Pub. No.: US 2007/0154577 A1
(43) Pub. Date: Jul. 5, 2007

(54) Herbal compositions inhibiting free radicals

(75) Inventors: Lie-Ching Row, Hsinchu City (TW); Chin-Chun Chen, Taipei County (TW); Lien Tai Chen, Taoyuan (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/432,372

(22) Filed: May 12, 2006

(30) Foreign Application Priority Data
Dec. 30, 2005 (TW) 94147657

(51) Int. Cl. A61K 36/00; A01N 65/00

(52) U.S. Cl. 424/774; 424/725

(57) ABSTRACT

An herbal composition. The herbal composition capable of inhibition of free radicals includes one of Polygonum cuspidatum and Viola yedoensis in an effective amount. The herbal composition is suitable for mammalian subjects. The Polygonum cuspidatum is selected from fresh Polygonum cuspidatum herbage, and the Viola yedoensis is selected from fresh Viola yedoensis herbage.

### BACKGROUND OF THE INVENTION

1. Field of the Invention

The invention relates to an herbal composition, and in particular relates to an herbal composition capable of inhibition of free radicals.

2. Description of the Related Art

Active free radicals may easily react with cells and DNA, resulting in aging and cancer.

Anti-oxidants such as vitamin E, vitamin C, carotenoids, trace elements, flavonoids, or phenol substances can effectively inhibit lipid peroxidation induced by free radicals. Anti-oxidants are cataloged into free radical terminators, reductants, chelating agents, oxygen scavengers, enzyme-type anti-oxidants, and peroxide decomposition agents. For human beings, anti-oxidants are important to resist disease.

Currently, various synthetic compounds capable of inhibition of free radicals have been developed. Most of them, however, result in environmental pollution. Thus, development of a natural anti-oxidant is important. Sources of natural anti-oxidants comprise vegetables, plants, herbal medicines, or fermentative foods.

### BRIEF SUMMARY OF THE INVENTION

The invention provides an herbal composition capable of inhibition of free radicals comprising one of Polygonum cuspidatum and Viola yedoensis in an effective amount.

A detailed description is given in the following embodiments

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides an herbal composition capable of inhibition of free radicals comprising one of Polygonum cuspidatum and Viola yedoensis in an effective amount. The Polygonum cuspidatum has concentration of about 0.01˜10 wt %, preferably 0.01˜1 wt %. The Viola yedoensis has concentration of about 0.01˜10 wt %, preferably 0.01˜1 wt %. The invention further provides an herbal composition capable of inhibition of free radicals comprising a mixture of Polygonum cuspidatum and Viola yedoensis in an effective amount, respectively. In the mixture, the Polygonum cuspidatum has concentration of about 0.01˜10 wt %, preferably 0.01˜1 wt %, and the Viola yedoensis has concentration of about 0.01˜10 wt %, preferably 0.01˜1 wt %.

The herbal composition is suitable for mammalian subjects, preferably human beings. The Polygonum cuspidatum is selected from fresh Polygonum cuspidatum herbage, and the Viola yedoensis is selected from fresh Viola yedoensis herbage.

After extraction, the herbal composition can be dispersed in water, ethanol, or ethyl acetate, preferably water. Further, the liquid herbal composition may be freeze-dried to form powder.

The herbal composition containing Polygonum cuspidatum has a free radical inhibition rate of about 90˜100%. The herbal composition containing Viola yedoensis has a free radical inhibition rate of about 90˜100%. The herbal composition containing a mixture of Polygonum cuspidatum and Viola yedoensis has a free radical inhibition rate of about 80˜100%.

The herbal composition may be oral or external, but not limited thereto. The oral herbal composition may comprise health food. The external herbal composition may comprise cosmetic such as treatment mask, lotion, or gel.

### EXAMPLE 1

600 g Polygonum cuspidatum herbage was thermal-extracted with 2L ethanol at 60° C. for 4 hours and repeated 2˜3 times. The extract solution was then re-concentrated to 200 ml. Next, 800 ml water and 300 ml n-hexene were added and extracted 3 times to form an aqueous layer and a n-hexene layer. The n-hexene layer was then re-concentrated (60° C., 2 hours) to form extracts thereof.

Next, the aqueous layer was extracted with 300 ml dichloromethane 3 times to form an aqueous layer and a dichloromethane layer. The dichloromethane layer was then re-concentrated (35° C., 2 hours) to form extracts thereof.

Next, the aqueous layer was extracted with 300 ml n-butanol 3 times to form an aqueous layer and a n-butanol layer. The n-butanol layer was then re-concentrated (70° C., 2 hours) to form extracts thereof. The last aqueous layer contained Polygonum cuspidatum herbal composition. The Polygonum cuspidatum had concentration of 2000 μg/ml.

Finally, the aqueous layer was re-concentrated (80° C., 2 hours) and freeze-dried for 3 days to form powder. The herbal composition powder was stored at −20° C.

Free Radical Inhibition Rate Experiment 0.05 ml Polygonum cuspidatum extract solution and 0.05 ml ethanol were, respectively, added to 0.2 ml solution containing 10 mM ethylene diamine tetraacetic acid (EDTA), 1% gelatin, 0.2 mg/ml poly(alpha-methylstyrene) (PMS), 4 mg/ml nitroblue tetrazolium (NBT), 1 mM xanthine, and 0.1M phosphate buffer solution (pH 7.8) at constant temperature of 25° C. Next, 0.05 ml xanthine oxidase solution and 0.05 ml phosphate buffer solution (0.1M, pH 7.8) were, respectively, added to the Polygonum cuspidatum-containing solution and ethanol-containing solution. After mixing completely, the absorption at 540 nm of various solutions was measured by ELISA reader. The absorption is represented by A, B, C, and D. A represents the absorption of the solution containing Polygonum cuspidatum and xanthine oxidase. B represents the absorption of the solution containing Polygonum cuspidatum and phosphate buffer solution. C represents the absorption of the solution containing ethanol and xanthine oxidase. D represents the absorption of the solution containing ethanol and phosphate buffer solution.

The free radical inhibition rate of the Polygonum cuspidatum extract solution was obtained from the following formula.

$$Free radical inhibition rate = \frac{( C - D ) - ( A - B )}{( C - D )} \times 100 \%$$

### EXAMPLE 2

600 g Viola yedoensis herbage was thermal-extracted with 2L ethanol at 60° C. for 4 hours and repeated 2˜3 times. The extract solution was then re-concentrated to 200 ml. Next, 800 ml water and 300 ml n-hexene were added and extracted 3 times to form an aqueous layer and a n-hexene layer. The n-hexene layer was then re-concentrated (60° C., 2 hours) to form extracts thereof.

Next, the aqueous layer was extracted with 300 ml dichloromethane 3 times to form an aqueous layer and a dichloromethane layer. The dichloromethane layer was then re-concentrated (35° C., 2 hours) to form extracts thereof.

Next, the aqueous layer was extracted with 300 ml n-butanol 3 times to form an aqueous layer and a n-butanol layer. The n-butanol layer was then re-concentrated (70° C., 2 hours) to form extracts thereof. The last aqueous layer contained Viola yedoensis herbal composition. The Viola yedoensis had concentration of 2000 μg/ml.

Finally, the aqueous layer was re-concentrated (80° C., 2 hours) and freeze-dried for 3 days to form powder. The herbal composition powder was stored at −20° C.

Free Radical Inhibition Rate Experiment

0.05 ml Viola yedoensis extract solution and 0.05 ml ethanol were, respectively, added to 0.2 ml solution containing 10 mM ethylene diamine tetraacetic acid (EDTA), 1% gelatin, 0.2 mg/ml poly(alpha-methylstyrene) (PMS), 4 mg/ml nitroblue tetrazolium (NBT), 1 mM xanthine, and 0.1M phosphate buffer solution (pH 7.8) at constant temperature of 25° C. Next, 0.05 ml xanthine oxidase solution and 0.05 ml phosphate buffer solution (0.1M, pH 7.8) were, respectively, added to the Viola yedoensis -containing solution and ethanol-containing solution. After mixing completely, the absorption at 540 nm of various solutions was measured by ELISA reader. The absorption is represented by A, B, C, and D. A represents the absorption of the solution containing Viola yedoensis and xanthine oxidase. B represents the absorption of the solution containing Viola yedoensis and phosphate buffer solution. C represents the absorption of the solution containing ethanol and xanthine oxidase. D represents the absorption of the solution containing ethanol and phosphate buffer solution.

The free radical inhibition rate of the Viola yedoensis extract solution was obtained from the following formula.

$$Free radical inhibition rate = \frac{( C - D ) - ( A - B )}{( C - D )} \times 100 \%$$

### EXAMPLE 3

600 g Polygonum cuspidatum herbage and 600 g Viola yedoensis herbage were thermal-extracted with 2L ethanol at 60° C. for 4 hours and repeated 2˜3 times. The extract solution was then re-concentrated to 200 ml. Next, 800 ml water and 300 ml n-hexene were added and extracted 3 times to form an aqueous layer and a n-hexene layer. The n-hexene layer was then re-concentrated (60° C., 2 hours) to form extracts thereof.

Next, the aqueous layer was extracted with 300 ml dichloromethane 3 times to form an aqueous layer and a dichloromethane layer. The dichloromethane layer was then re-concentrated (35° C., 2 hours) to form extracts thereof.

Next, the aqueous layer was extracted with 300 ml n-butanol 3 times to form an aqueous layer and a n-butanol layer. The n-butanol layer was then re-concentrated (70° C., 2 hours) to form extracts thereof. The last aqueous layer contained Polygonum cuspidatum and Viola yedoensis herbal compositions.

Finally, the aqueous layer was re-concentrated (80° C., 2 hours) and freeze-dried for 3 days to form powder. The herbal composition powder was stored at −20° C.

Free Radical Inhibition Rate Experiment 0.05 ml extract solution containing Polygonum cuspidatum and Viola yedoensis and 0.05 ml ethanol were, respectively, added to 0.2 ml solution containing 10 mM ethylene diamine tetraacetic acid (EDTA), 1% gelatin, 0.2 mg/ml poly(alpha-methylstyrene) (PMS), 4 mg/ml nitroblue tetrazolium (NBe, lmM xanthine, and 0.1M phosphate buffer solution (pH 7.8) at constant temperature of 25° C. Next, O.05 ml xanthine oxidase solution and 0.05 ml phosphate buffer solution (0.1M, pH 7.8) were, respectively, added to the solution containing Polygonum cuspidatum and Viola yedoensis and the ethanol-containing solution. After mixing completely, the absorption at 540 nm of various solutions was measured by ELISA reader. The absorption is represented by A, B, C, and D. A represents the absorption of the solution containing Polygonum cuspidatum, Viola yedoensis , and xanthine oxidase. B represents the absorption of the solution containing Polygonum cuspidatum, Viola yedoensis , and phosphate buffer solution. C represents the absorption of the solution containing ethanol and xanthine oxidase. D represents the absorption of the solution containing ethanol and phosphate buffer solution.

The free radical inhibition rate of the extract solution containing Polygonum cuspidatum and Viola yedoensis was obtained from the following formula.

$$Free radical inhibition rate = \frac{( C - D ) - ( A - B )}{( C - D )} \times 100 \%$$

### EXAMPLE 4

The example provides a health food formed by the herbal composition. The content thereof comprises plant extracts (Polygonum cuspidatum and Viola yedoensis ), cellulose (avicel 102), dicalcium phosphate, lactose, and corn starch.

### EXAMPLE 5

The example provides a lotion formed by the herbal composition. The content thereof comprises deionized water, xanthan gum, hydrogenated polyisobutene, isopropyl isostearate, glycerin, 1,3-butylene glycol, cestearyl alcohol, hyaluronic acid, steareth-21, steareth-2, dimethicone, plant extracts (Polygonum cuspidatum and Viola yedoensis), tocopheryl acetate, retinyl palmitate, 2-phenoxyethanol, imidazolidinyl urea, propylparaben, methyparaben, disodium EDTA, and fragrance.

Skin Analysis

30˜50 health women applied the lotion to their left side faces and kept the other side clean. Skin was analyzed by various instruments every week until a ₅^{th} week. In this analysis, improvement of skin quality was estimated from water content of cuticle, water-loss rate, elasticity, sebum secretion, black spot, pore number, and melanin.

The analytical results are recited in Table 1. The results indicate that the lotion provided by the invention effectively reduced black spot, pore number, melanin, water-loss rate, and sebum secretion, and increase water content of cuticle and skin elasticity.

TABLE 1

| | | | | Water | Water- | |
|---|---|---|---|---|---|---|
| Black | Pore | | | content | loss | Sebum |
| spot | number | Melanin | Elasticity | of cuticle | rate | secretion |
| −15.0% | −12.6% | −22.3% | +3.0% | +30% | −8% | −2% |

While the invention has been described by way of example and in terms of the preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An herbal composition of inhibiting free radical comprising an effective amount of Polygonum cuspidatum.

2. The herbal composition of inhibiting free radical as claimed in claim 1, wherein the herbal composition is powder.

3. The herbal composition of inhibiting free radical as claimed in claim 1, wherein the herbal composition is dispersed in water, ethanol, or ethyl acetate.

4. The herbal composition of inhibiting free radical as claimed in claim 3, wherein the Polygonum cuspidatum has concentration of about 0.01˜10 wt %.

5. The herbal composition of inhibiting free radical as claimed in claim 1, wherein the herbal composition has a free radical inhibition rate of about 90˜100%.

6. The herbal composition of inhibiting free radical as claimed in claim 1, wherein the herbal composition is oral or external.

7. The herbal composition of inhibiting free radical as claimed in claim 6, wherein the oral herbal composition comprises health food.

8. The herbal composition of inhibiting free radical as claimed in claim 6, wherein the external herbal composition comprises cosmetic.

9. The herbal composition of inhibiting free radical as claimed in claim 8, wherein the cosmetic reduces skin melanin of about 10˜30%.

10. The herbal composition of inhibiting free radical as claimed in claim 8, wherein the cosmetic increases skin elasticity of about 1˜5%.

11. The herbal composition of inhibiting free radical as claimed in claim 8, wherein the cosmetic increases water content of cuticle of about 20˜40%.

12. An herbal composition of inhibiting free radical comprising an effective amount of Viola yedoensis.

13. The herbal composition of inhibiting free radical as claimed in claim 12, wherein the herbal composition is powder.

14. The herbal composition of inhibiting free radical as claimed in claim 12, wherein the herbal composition is dispersed in water, ethanol, or ethyl acetate.

15. The herbal composition of inhibiting free radical as claimed in claim 14, wherein the Viola yedoensis has concentration of about 0.01˜10 wt %.

16. The herbal composition of inhibiting free radical as claimed in claim 12, wherein the herbal composition has a free radical inhibition rate of about 90˜100%.

17. The herbal composition of inhibiting free radical as claimed in claim 12, wherein the herbal composition is oral or external.

18. The herbal composition of inhibiting free radical as claimed in claim 17, wherein the oral herbal composition comprises health food.

19. The herbal composition of inhibiting free radical as claimed in claim 17, wherein the external herbal composition comprises cosmetic.

20. The herbal composition of inhibiting free radical as claimed in claim 19, wherein the cosmetic reduces skin melanin of about 10˜30%.

21. The herbal composition of inhibiting free radical as claimed in claim 19, wherein the cosmetic increases skin elasticity of about 1˜5%.

22. The herbal composition of inhibiting free radical as claimed in claim 19, wherein the cosmetic increases water content of cuticle of about 20˜40%.
